(19) United States
(12) Patent Application Publication
(10) Pub. No.: US 2005/0158374 A1
(43) Pub. Date: Jul. 21, 2005

(54) Compositions and dosage forms for enhanced absorption of metformin

(76) Inventors: Patrick S.L. Wong, Burlingame, CA (US); Dong Yan, Cupertino, CA (US); George V. Guittard, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/978,141

(22) Filed: Oct. 29, 2004

Related U.S. Application Data
(60) Provisional application No. 60/516,259, filed on Oct. 31, 2003.
(60) Provisional application No. 60/519,509, filed on Nov. 12, 2003.

(51) Int. Cl. A61K 9/127; A61K 31/205; A61K 31/155

(52) U.S. Cl. 424/450; 514/554; 514/635

(57) ABSTRACT

A complex comprised of metformin and a transport moiety, such as a fatty acid, is described. The complex has an enhanced absorption in the gastrointestinal tract, particularly the lower gastrointestinal tract. The complex, and compositions and dosage forms prepared using the complex, provide for absorption by the body of the drug through a period of ten to twenty-four hours, thus enabling a once-daily dosage form for metformin.

### CROSS-REFERENCE TO RELATED APPLICATIONS

This application claims the benefit of U.S. provisional patent application No. 60/516,259, filed Oct. 31, 2003, and of U.S. provisional patent application No. 60/519,509, filed Nov. 12, 2003, both applications are incorporated herein by reference in their entirety.

### FIELD OF THE INVENTION

This invention relates to the compositions and dosage forms for delivery of metformin. More particularly, the invention relates to a complex of metformin and a transport moiety where the complex provides an enhanced absorption of metformin in the gastrointestinal tract, and more particularly, in the lower gastrointestinal tract.

### BACKGROUND OF THE INVENTION

Conventional pharmaceutical development of dosage forms is based on obtaining, on the one hand, a stable dosage form, and on the other hand, a dosage form that maximizes absorption in the upper gastrointestinal tract. Since most drug dosage forms are designed for immediate release of the drug, the dosage form is designed to be well dissolved in the upper gastrointestinal (G.I.) tract because the upper G.I. tract has a far greater surface area for absorption of drugs than does the lower G.I. tract. The lower G.I. tract, or colon, lacks microvilli which are present in the upper G.I. tract. The presence of microvilli greatly increases the surface area for drug absorption, and the upper G.I. tract has 480 times the surface area than does the colon.

Differences in the cellular characteristics of the upper and lower G.I. tracts also contribute to the poor absorption of molecules in the lower G.I. tract. FIG. 1 illustrates two common routes for transport of compounds across the epithelium of the G.I. tract. Individual epithelial cells, represented by 10a, 10b, 10c, form a cellular barrier along the small and large intestine. Individual cells are separated by water channels or tight junctions, such as junctions 12a, 12b. Transport across the epithelium occurs via either or both a transcellular pathway and a paracellular pathway. The transcellular pathway for transport, indicated in FIG. 1 by arrow 14, involves movement of the compound across the wall and body of the epithelial cell by passive diffusion or by carrier-mediated transport. The paracellular pathway of transport involves movement of molecules through the tight junctions between individual cells, as indicated by arrow 16. Paracellular transport is less specific but has a much greater overall capacity, in part because it takes place throughout the length of the G.I. tract. However, the tight junctions vary along the length of the G.I. tract, with an increasing proximal to distal gradient in effective ‘tightness’ of the tight junction. Thus, the duodenum in the upper G.I. tract is more “leaky” than the ileum in the upper G.I. tract which is more “leaky” than the colon in the lower G.I. tract (Knauf, H. et al., Klin. Wochenschr., 60(19):1191-1200 (1982)).

Since the typical residence time of a drug in the upper G.I. tract is from approximately four to six hours, drugs having poor colonic absorption are absorbed by the body through a period of only four to six hours after oral ingestion. Frequently it is medically desirable that the administered drug be presented in the patient's blood stream at a relatively constant concentration throughout the day. To achieve this with traditional drug formulations that exhibit minimal colonic absorption, patients would need to ingest the drugs three to four times a day. Practical experience with this inconvenience to patients suggests that this is not an optimum treatment protocol. Accordingly, it is desired that a once daily administration of such drugs, with long-term absorption throughout the day, be achieved.

To provide constant dosing treatments, conventional pharmaceutical development has suggested various controlled release drug systems. Such systems function by releasing their payload of drugs over an extended period of time following administration. However, these conventional forms of controlled release systems are not effective in the case of drugs exhibiting minimal colonic absorption. Since the drugs are only absorbed in the upper G.I. tract and since the residence time of the drug in the upper G.I. tract is only four to six hours, the fact that a proposed controlled release dosage form may release its payload after the residence period of the dosage form in the upper G.I. does not mean the that body will continue to absorb the controlled release drug past the four to six hours of upper G.I. residence. Instead, the drug released by the controlled release dosage form after the dosage form has entered the lower G.I. tract is generally not absorbed and, instead, is expelled from the body with other matter from the lower G.I.

Metformin is a compound established to have poor colonic absorption (Marathe, P. et al., Br. J. Clin. Pharmacol., 50:325-332 (2000)). Metformin hydrochloride has an intrinsically poor permeability and absorption in the lower G.I. tract, or the colon, leading to absorption almost exclusively in the upper part of the gastrointestinal tract (upper G.I. tract).

Metformin is an anti-hyperglycemic agent of the biguanide class used in the treatment of type II diabetes. It is commercially available as a hydrochloride salt, metformin HCl, and is marketed as Glucophage® for treatment of non-insulin-dependent diabetes mellitus (type II diabetes). For patients with diabetes, a once-daily metformin treatment would provide advantages beyond convenience, since pharmacodynamic advantages are provided by a relatively constant dosage of metformin in the bloodstream. For example, a relatively constant dosage could improve glucose utilization and glucose tolerance.

Prior art approaches to sustained release of metformin have focused on increasing the residence time in the upper G.I. tract. For example, PCT Publication WO 99/47128 describes metformin delivery systems which are predicated on the principle that absorption of metformin occurs primarily only in the upper G.I. tract, and not in the lower G.I. tract. Other prior art attempts to formulate a once-daily dose for metformin have been largely unsuccessful. For example, Glucophage XR® is proposed as a once-daily formulation, but releases 90% of its dose in about six hours in vitro, far less than a proposed 15 to 20 hour once-daily dosage form. Thus, twice daily dosing of Glucophage XR® is required. Others have proposed an extended release dosage form of metformin HCl, where absorption time in the stomach is extended by increasing the retention time of the dosage form in the stomach (U.S. Pat. No. 6,451,808; U.S. Pat. No 6,723,340).

Thus, there remains a need for a once-daily metformin dosage system, where the dosage system provides absorption of metformin in the lower G.I. tract.

### SUMMARY OF THE INVENTION

According, in one aspect, the invention includes a substance comprised of metformin and a transport moiety, the metformin and the transport moiety forming a complex.

In one embodiment, the transport moiety, prior to complex formation, is a fatty acid of the form CH₃(CₙH₂ₙ)COOH, where n is from 4-16.

In another embodiment, the transport moiety is capric acid or lauric acid.

In another aspect, the invention includes a composition, comprising, a complex comprised of metformin and a transport moiety, and a pharmaceutically acceptable vehicle, wherein the composition has an absorption in the lower gastrointestinal tract at least four-fold higher than metformin hydrochloride.

In another aspect, the invention includes a dosage form comprising the composition described above.

In another aspect, the invention includes a dosage form comprising the substance described above.

In various embodiment, the dosage form is an osmotic dosage form.

An exemplary dosage form is one comprised of (i) a push layer; (ii) drug layer comprising a metformin-transport moiety complex; (iii) a semipermeable wall provided around the push layer and the drug layer; and (iv) an exit.

Another exemplary dosage form is one comprised of (i) a semipermeable wall provided around an osmotic formulation comprising a metformin-transport moiety complex, an osmagent, and an osmopolymer; and (ii) an exit.

In one embodiment, the dosage form provides a total daily dose of between 500-2550 mg.

In another aspect, the invention provides an improvement in a dosage form comprising metformin or a salt of metformin. The improvement comprises a dosage form including of a complex of metformin and a transport moiety.

In another aspect, the invention includes a method for treating hyperglycemia in a subject, comprising administering the composition described above.

In one embodiment, the composition is administered orally.

In another aspect, the invention includes a method of preparing a metformin-transport moiety complex, comprising providing metformin base; providing a transport moiety; combining the metformin base and the transport moiety in the presence of a solvent having a dielectric constant less than that of water; whereby combining forms a complex between the metformin base and the transport moiety.

In one embodiment, metformin and the transport moiety are combined in a solvent having a dielectric constant at least two fold lower than the dielectric constant of water. Exemplary solvents are methanol, ethanol, acetone, benzene, methylene chloride, and carbon tetrachloride.

In another aspect, the invention includes a method of improving gastrointestinal absorption of metformin, comprising providing a complex comprised of metformin and a transport moiety, said complex characterized by a tight-ion pair bond; and administering the complex to a patient.

In one embodiment, the improved absorption comprises improved lower gastrointestinal absorption.

In another embodiment, the improved absorption comprises improved absorption in the upper gastrointestinal tract.

In another aspect, the invention includes a method of treating a subject having Type II diabetes, comprising administering a complex comprised of metformin and a transport moiety; administering a second therapeutic agent.

In one embodiment, administration of a second therapeutic agent comprises administering a second therapeutic agent that is an anti-diabetic agent.

In another embodiment, the second therapeutic agent is a dipeptidyl peptidase IV inhibitor.

In yet another embodiment, the complex of metformin and a fatty acid transport moiety is comprised of a fatty acid where prior to complex formation, the fatty acid is of form CH₃(CₙH₂ₙ)COOH, where n is from 4-16. Exemplary fatty acids are capric acid or lauric acid.

In another embodiment, the complex and/or the DPP IV inhibitor is orally administered.

In yet another aspect, the invention includes a compound comprising metformin and a transport moiety, the compound prepared by a process of (i) providing metformin base; (ii) providing a transport moiety; (iii) combining the metformin base and the transport moiety in the presence of a solvent having a dielectric constant less than that of water, where the combining forms a complex between the metformin base and the transport moiety associated by a tight-ion pair bond.

These and other objects and features of the invention will be more fully appreciated when the following detailed description of the invention is read in conjunction with the accompanying drawings.

### BRIEF DESCRIPTION OF THE FIGURES

The following figures are not drawn to scale, and are set forth to illustrate various embodiments of the invention.

FIG. 1 is a diagram of epithelial cells of the gastrointestinal tract, illustrating the transcellular pathway and the paracellular pathway for transport of molecules through the epithelium;

FIG. 2 shows the chemical structure of metformin;

FIG. 3 is a plot of the logarithm of the octanol/water partition coefficient as a function of pH for metformin HCl;

FIG. 4A shows a generalized synthetic reaction scheme for preparation of a metformin-transport moiety complex;

FIG. 4B shows a generalized synthetic reaction scheme for preparation of a metformin-transport moiety complex, where the transport moiety includes a carboxyl group;

FIG. 4C shows a synthetic reaction scheme for preparation of a metformin-fatty acid complex;

FIGS. 5A-5D are HPLC traces of metformin HCl (FIG. 5A), sodium laurate (FIG. 5B), and a physical mixture of metformin HCl, sodium laurate (FIG. 5C), and metformin-laurate complex (FIG. 5D);

FIGS. 6A-6B are plots of conductivity, in microsiemens/centimeter (μS/cm, FIG. 6A) and percent of non-ionized drug (FIG. 6B), as a function of metformin concentration for metformin HCl (circles), metformin complexed with succinate (inverted triangles), caprate (squares), laurate (diamonds), palmitate (triangles), and oleate (octagons);

FIG. 7 shows the metformin plasma concentration, in ng/mL, in rats as a function of time, in hours, for metformin HCl (circles) and a metformin-laurate complex (diamonds) following oral gavage of the compounds to rats;

FIG. 8 shows the metformin plasma concentration, in ng/mL, in rats as a function of time, in hours, for metformin HCl (circles), metformin complexed with succinate (diamonds), palmitate (triangles), oleate (inverted triangles), caprate (squares), and laurate (octagons), using a flush-ligated colonic model;

FIG. 9 shows the percent bioavailability as a function of metformin dose, in mg base/kg, of a physical mixture of metformin HCl and sodium laurate (circles) and of a metformin laurate complex (squares) in rat plasma using a flush-ligated colonic model;

FIG. 10 is a plot of metformin base plasma concentration, in ng/mL, in rats as a function of time, in hours, following intravenous administration of 2 mg/kg metformin hydrochloride (triangles) and following administration of a 10 mg/rat dose of metformin hydrochloride (circles) or metformin laurate complex (diamonds) using a flushed ligated colonic model;

FIG. 11 illustrates an exemplary osmotic dosage form shown in cutaway view,

FIG. 12 illustrates another exemplary osmotic dosage form for a once daily dosing of metformin, the dosage form comprising a metformin-transport moiety complex with an optional loading dose of the complex in the outer coating;

FIG. 13A illustrates one embodiment of a once daily metformin dosage form comprising both metformin HCl and a metformin-laurate complex, with an optional loading dose of metformin HCl by coating;

FIG. 13B is a bar graph showing the release rate of metformin, in mg/hour, as a function of time, in hours, of a 300 mg metformin hydrochloride equivalent dose from the dosage form of FIG. 13A;

FIGS. 14A-14C illustrate an embodiment of a dosage prior to administration to a subject and comprising a complex of metformin-transport moiety complex in a matrix (FIG. 14A), in operation after ingestion into the gastrointestinal tract (FIG. 14B), and after sufficient erosion of the matrix has caused separation of the banded sections of the device (FIG. 14C).

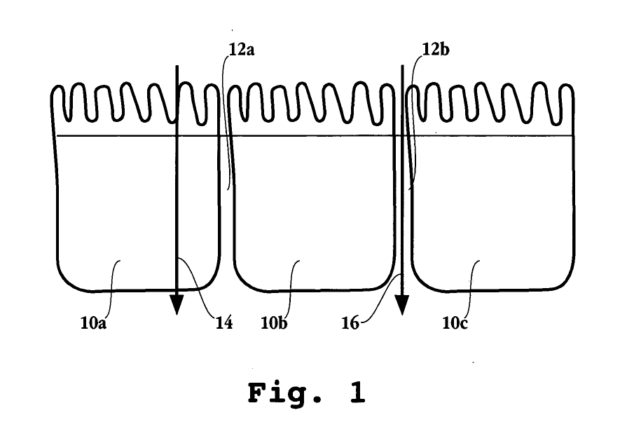

### DETAILED DESCRIPTION

I. Definitions

The present invention is best understood by reference to the following definitions, the drawings and exemplary disclosure provided herein.

By “composition” is meant one or more of metformin-transport moiety complexes, optionally in combination with additional active pharmaceutical ingredients, and/or optionally in combination with inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like.

By “complex” is meant a substance comprising a drug moiety (e.g., metformin) and a transport moiety associated by a tight-ion pair bond. A drug-moiety-transport moiety complex can be distinguished from a loose ion pair of the drug moiety and the transport moiety by a difference in octanol/water partitioning behavior, characterized by the following relationship:
ΔLogD=Log D(complex)−Log D(loose-ion pair)≧0.15 (Equation 1)
wherein, D, the distribution coefficient (apparent partition coefficient), is the ratio of the equilibrium concentrations of all species of the drug moiety and the transport moiety in octanol to the same species in water (deionized water) at a set pH (typically about pH=5.0 to about pH=7.0) and at 25 degrees Celsius. Log D (complex) is determined for a complex of the drug moiety and transport moiety prepared according to the teachings herein. Log D (loose-ion pair) is determined for a physical mixture of the drug moiety and the transport moiety in deionized water. For instance, the octanol/water apparent partition coefficient (D=C_{octanol}/C_{water}) of a putative complex (in deionized water at 25 degree Celsuis) can be determined and compared to a 1:1 (mol/mol) physical mixture of the transport moiety and the drug moiety in deionized water at 25 degree Celsuis. If the difference between the Log D for the putative complex (D+T−) and the Log D for the 1:1 (mol/mol) physical mixture, D⁺∥T⁻ is determined is greater than or equal to 0.15, the putative complex is confirmed as being a complex according to the invention. In preferable embodiments, ΔLog D≧0.20, and more preferably ΔLog D≧0.25, more preferably still ΔLog D≧0.35.

By “dosage form” is meant a pharmaceutical composition in a medium, carrier, vehicle, or device suitable for administration to a patient in need thereof.

By “drug” or “drug moiety” is meant a drug, compound, or agent, or a residue of such a drug, compound, or agent that provides some pharmacological effect when administered to a subject. For use in forming a complex, the drug comprises a(n) acidic, basic, or zwitterionic structural element, or a(n) acidic, basic, or zwitterionic residual structural element.

By “fatty acid” is meant any of the group of organic acids of the general formula CH₃(CₙHₓ)COOH where the hydrocarbon chain is either saturated (x=2n, e.g. palmitic acid, C₁₅H₃₁COOH) or unsaturated (x=2n−2, e.g. oleic acid, CH₃C₁₆H₃₀COOH).

By “intestine” or “gastrointestinal (G.I.) tract” is meant the portion of the digestive tract that extends from the lower opening of the stomach to the anus, composed of the small intestine (duodenum, jejunum, and ileum) and the large intestine (ascending colon, transverse colon, descending colon, sigmoid colon, and rectum).

By “loose ion-pair” is meant a pair of ions that are, at physiologic pH and in an aqueous environment, are readily interchangeable with other loosely paired or free ions that may be present in the environment of the loose ion pair. Loose ion-pairs can be found experimentally by noting interchange of a member of a loose ion-pair with another ion, at physiologic pH and in an aqueous environment, using isotopic labeling and NMR or mass spectroscopy. Loose ion-pairs also can be found experimentally by noting separation of the ion-pair, at physiologic pH and in an aqueous environment, using reverse phase HPLC. Loose ion-pairs may also be referred to as “physical mixtures,” and are formed by physically mixing the ion-pair together in a medium.

By “lower gastrointestinal tract” or “lower G.I. tract” is meant the large intestine.

Metformin refers to N,N-dimethylimidodicarbonimidic diamide, and has a molecular formula of C₄H₁₁N₅, molecular weight of 129.17. The compound is commercially available as metformin hydrochloride.

By “patient” is meant an animal, preferably a mammal, more preferably a human, in need of therapeutic intervention.

By “tight-ion pair” is meant a pair of ions that are, at physiologic pH and in an aqueous environment are not readily interchangeable with other loosely paired or free ions that may be present in the environment of the tight-ion pair. A tight-ion pair can be experimentally detected by noting the absence of interchange of a member of a tight ion-pair with another ion, at physiologic pH and in an aqueous environment, using isotopic labeling and NMR or mass spectroscopy. Tight ion pairs also can be found experimentally by noting the lack of separation of the ion-pair, at physiologic pH and in an aqueous environment, using reverse phase HPLC.

By “transport moiety” is meant a compound that is capable of forming, or a residue of that compound that has formed, a complex with a drug, wherein the transport moiety serves to improve transport of the drug across epithelial tissue, compared to that of the uncomplexed drug. The transport moiety comprises a hydrophobic portion and a(n) acidic, basic, or zwitterionic structural element, or a(n) acidic, basic, or zwitterionic residual structural element. In a preferred embodiment, the hydrophobic portion comprises a hydrocarbon chain. In an embodiment, the pKa of a basic structural element or basic residual structural element is greater than about 7.0, preferably greater than about 8.0.

By “pharmaceutical composition” is meant a composition suitable for administration to a patient in need thereof.

By “structural element” is meant a chemical group that (i) is part of a larger molecule, and (ii) possesses distinguishable chemical functionality. For example, an acidic group or a basic group on a compound is a structural element.

By “substance” is meant a chemical entity having specific characteristics.

By “residual structural element” is meant a structural element that is modified by interaction or reaction with another compound, chemical group, ion, atom, or the like. For example, a carboxyl structural element (COOH) interacts with sodium to form a sodium-carboxylate salt, the COO— being a residual structural element.

By “upper gastrointestinal tract” or “upper G.I. tract” is meant that portion of the gastrointestinal tract including the stomach and the small intestine.

II. Metformin Complex Formation and Characterization

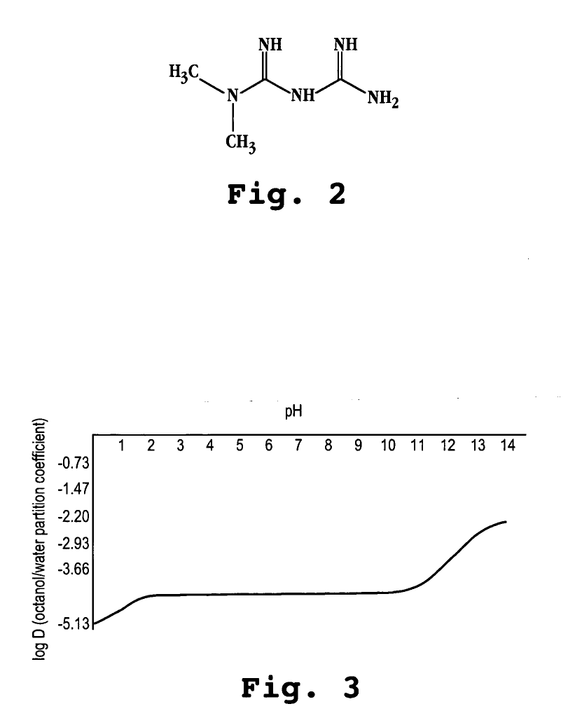

As noted above, metformin is an anti-hyperglycemic agent of the biguanide class used to help control blood sugar levels in non-insulin-dependent diabetes mellitus (type II diabetes). Metformin, shown in FIG. 2, is a cationic, water-soluble compound with a pKa of 12.4. The ionized form of the drug tends to adsorb to the negatively-charged intestinal epithelium and studies have shown that metformin has poor colonic absorption in healthy human subjects (Vidon, N., et al, Diabetes Res. Clin. Pract., 4:223-229 (1988)). The hydrophilicity of metformin hydrochloride is shown in FIG. 3 where the logarithm of the octanol/water partition coefficient (logP) as a function of pH for metformin HCl is plotted. At pH values less than 7.0, metformin hydrochloride is hydrophilic, with a logP of less than −3.7. The pH gradient in the G.I. tract ranging from a pH of about 1.2 in the stomach to a pH of about 7.5 in the distal ileum and large intestine (Evans, D. F. et al., Gut, 29:1035-1041 (1988)) means that metformin hydrochloride is hydrophilic over the range of pH in the G.I. tract. Moreover, metformin HCl is highly dissociated at these pH values. The combination of hydrophilicity and charge tends to severely limit its absorption via transcellular pathways and as a result, metformin HCl is very poorly absorbed in the lower G.I. tract.

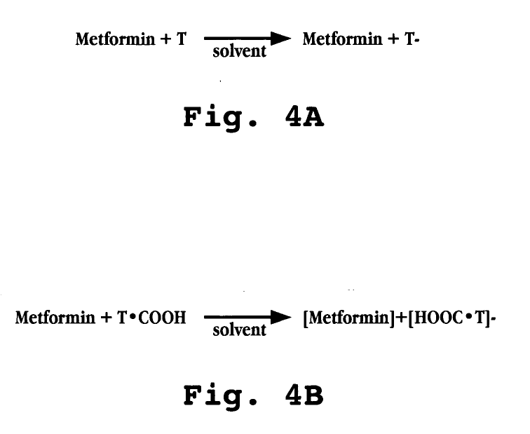

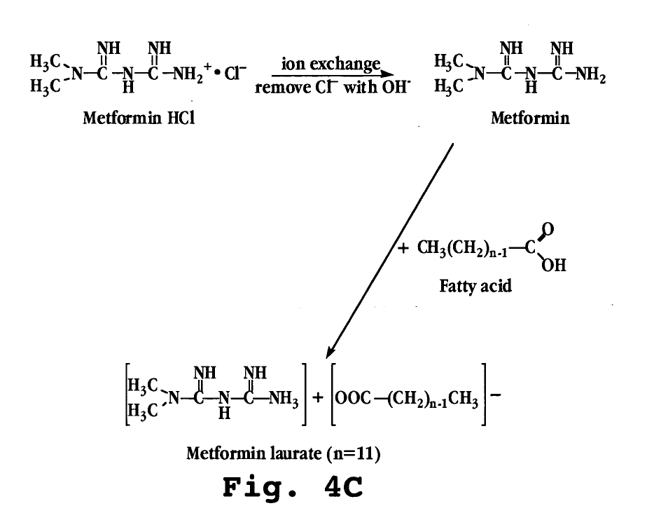

Accordingly, in one aspect, the invention provides a substance comprising metformin that has significantly improved absorption in the lower G.I. tract. The substance is a complex of metformin and a transport moiety and can be prepared from a salt of metformin, such as metformin hydrochloride, according to the generalized synthetic reaction scheme shown in FIG. 4A. Briefly, metformin is combined with a transport moiety, represented as T⁻M⁺ in the drawing. Exemplary transport moieties are listed above and include fatty acids, benzenesulfonic acid, benzoic acid, fumaric acid, and salicylic acid. The two species are contacted in the presence of an organic solvent that has a dielectric constant less than water, as will be discussed below, to form a metformin-transport moiety complex where the species are associated by tight-ion pair bond, as denoted in FIG. 4A by the representation Metformin+(T)−.

FIG. 4B illustrates a more specific synthetic reaction scheme for formation of a metformin-transport moiety complex. In this scheme, the transport moiety has a carboxyl group (COO⁻), represented as T-COO⁻ in the drawing. The carboxyl-containing transport moiety, T-COO⁻, is mixed in a solvent having a dielectric constant less than water, to form a complex of metformin and the transport moiety associated by a hybrid bond or a tight ion pair, denoted in the drawing as Metformin+[(T COO)₂]−.

A specific example of the procedure for preparing a metformin-transport moiety complex, where the transport moiety is a fatty acid, is provided in Example 1, and illustrated in FIG. 4C. Metformin base is prepared from the hydrochloride salt using an ion exchange process. A solution of a fatty acid in a solvent is contacted with metformin base to form recover the metformin-fatty acid complex.

In Example 1, a complex was formed from lauric acid as an exemplary fatty acid transport moiety. It will be understood that lauric acid is merely exemplary and that the preparation procedure is equally applicable to other species suitable as a transport moiety, and to fatty acids of any carbon chain length. For example, complex formation of metformin with various fatty acids or salts of fatty acids, the fatty acids having from 6 to 18 carbon atoms, more preferably 8 to 16 carbon atoms and even more preferably 10 to 14 carbon atoms. The fatty acids or their salts can be saturated or unsaturated. Exemplary saturated fatty acids contemplated for use in preparation of the complex include butanoic (butyric, 4C); pentanoic (valeric, 5C); hexanoic (caproic, 6C); octanoic (caprylic, 8C); nonanoic (pelargonic, 9C); decanoic (capric, 10C); dodecanoic (lauric, 12C); tetradecanoic (myristic, 14C); hexadecanoic (palmitic, 16C); heptadecanoic (margaric, 17C); and octadecanoic (stearic, 18C), where the systematic name is followed in parenthesis by the trivial name and the number of carbon atoms in the fatty acid. Unsaturated fatty acids include oleic acid, linoleic acid, and linolenic acid, all having 18 carbon atoms. Linoleic acid and linolenic acid are polyunsaturated.

Complex formation of metformin with alkyl sulfates or a salt of an alkyl sulfate is also contemplated, where the alkyl sulfate may be saturated or unsaturated. Exemplary alkyl sulfates, or their salts (sodium, potassium, magnesium, etc), have from 6 to 18 carbon atoms, more preferably 8 to 16 and even more preferably 10 to 14 carbon atoms. Complex formation of metformin with the benzenesulfonic acid, benzoic acid, fumaric acid, and salicylic acid, or the salts of these acids, is also contemplated.

In one embodiment, complexes in accord with the invention exclude a complex of metformin-thiocitic acid (also known as alpha-lipoic acid).

With continuing reference to Example 1, the complex consisting of metformin-laurate was prepared from acetone. Acetone is merely an exemplary solvent, and other solvents in which fatty acids are soluble are suitable. For example, fatty acids are soluble in chloroform, benzene, cyclohexane, ethanol (95%), acetic acid, and methanol. The solubility (in g/L) of capric acid, lauric acid, myristic acid, palmitic acid, and stearic acid in these solvents is indicated in Table 1.

TABLE 1
Solubility (g/L) of Fatty Acids at 20° C.

| Fatty Acid | | | | | Ethanol | Acetic | | |
|---|---|---|---|---|---|---|---|---|
| (no. carbons) | Chloroform | Benzene | Cyclohexanne | Acetone | 95% | acid | Methanol | Acetonitrile |
| Capric (10) | 3260 | 3980 | 3420 | 4070 | 4400 | 5670 | 5100 | 660 |
| Lauric (12) | 830 | 936 | 680 | 605 | 912 | 818 | 1200 | 76 |
| myristic (14) | 325 | 292 | 215 | 159 | 189 | 102 | 173 | 18 |
| palmitic (16) | 151 | 73 | 65 | 53.8 | 49.3 | 21.4 | 37 | 4 |
| stearic (18) | 60 | 24.6 | 24 | 15.4 | 11.3 | 1.2 | 1 | <1 |

In one embodiment, the solvent used for formation of the complex is a solvent having a dielectric constant less than water, and preferably at least two fold lower than the dielectric constant of water, more preferably at least three-fold lower than that of water. The dielectric constant is a measure of the polarity of a solvent and dielectric constants for exemplary solvents are shown in Table 2.

TABLE 2
Characteristics of Exemplary Solvents

| Solvent | Boiling Pt., ° C. | Dielectric constant |
|---|---|---|
| Water | 100 | 80 |
| Methanol | 68 | 33 |
| Ethanol | 78 | 24.3 |
| 1-propanol | 97 | 20.1 |
| 1-butanol | 118 | 17.8 |
| acetic acid | 118 | 6.15 |
| Acetone | 56 | 20.7 |
| methyl ethyl ketone | 80 | 18.5 |
| ethyl acetate | 78 | 6.02 |
| Acetonitrile | 81 | 36.6 |
| N,N-dimethylformamide (DMF) | 153 | 38.3 |
| dimethyl sulfoxide (DMSO) | 189 | 47.2 |
| Hexane | 69 | 2.02 |
| Benzene | 80 | 2.28 |
| diethyl ether | 35 | 4.34 |
| tetrahydrofuran (THF) | 66 | 7.52 |
| methylene chloride | 40 | 9.08 |
| carbon tetrachloride | 76 | 2.24 |

The solvents water, methanol, ethanol, 1-propanol, 1-butanol, and acetic acid are polar protic solvents having a hydrogen atom attached to an electronegative atom, typically oxygen. The solvents acetone, ethyl acetate, methyl ethyl ketone, and acetonitrile are dipolar aprotic solvents, and are in one embodiment, preferred for use in forming the metformin complex. Dipolar aprotic solvents do not contain an OH bond but typically have a large bond dipole by virtue of a multiple bond between carbon and either oxygen or nitrogen. Most dipolar aprotic solvents contain a C—O double bond. The dipolar aprotic solvents noted in Table 2 have a dielectric constant at least two-fold lower than water.

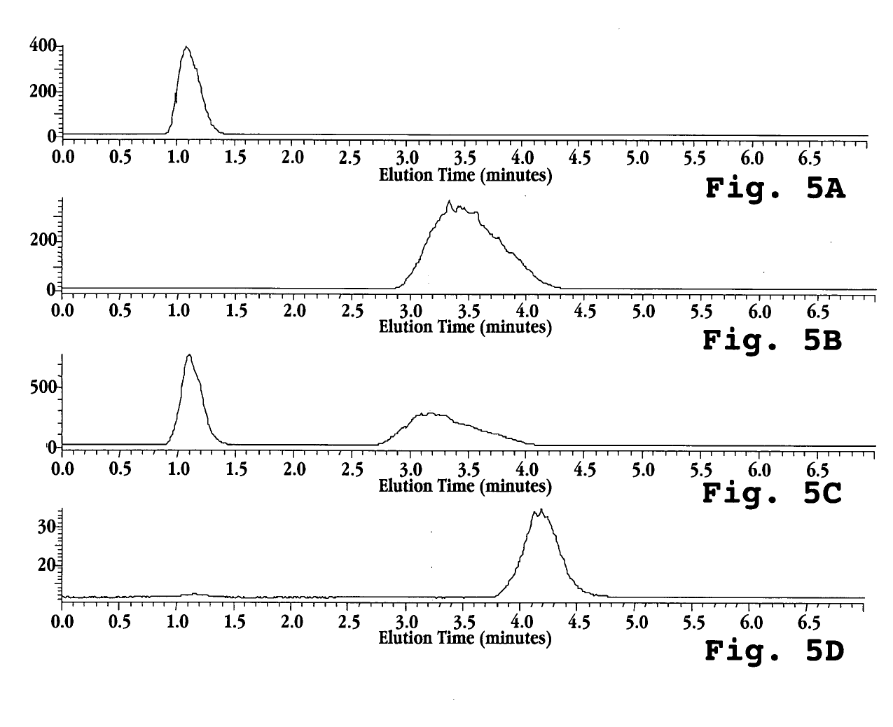

Reverse phase HPLC was used to analyze the metformin-laurate complex formed as described in Example 1. The HPLC conditions are described in the methods section below. For comparison, HPLC traces of metformin HCl, of sodium laurate, and of a physical mixture of metformin HCl and sodium laurate were also generated, and the results are shown in FIGS. 5A-5D. The trace for metformin hydrochloride is shown in FIG. 5A, and a single peak at 1.1 minutes is observed. The salt form of lauric acid, sodium laurate, elutes as a single, broad peak between about 3-4 minutes (FIG. 5B). A 1:1 molar physical mixture of metformin HCl and sodium laurate in water elutes as two peaks, one peak at 1.1 minutes corresponding to metformin hydrochloride and a second peak between about 2.7-4 minutes of sodium laurate (FIG. 5C). FIG. 5D shows the HPLC trace for the complex formed by the procedure in Example 2, where a single peak eluting between 3.9-4.5 minutes is observed. The HPLC traces show that the complex formed of metformin base and lauric acid is different from the physical mixture of the two components in water. The trace also shows that the complex does not dissociate when subjected to the solvent system (water:acetonitrile 50.50 v:v) for the HPLC analysis.

In another study to characterize the metformin-laurate complex, the octanol/water apparent partition coefficient (D=C_{octanil}/C_{water}) of the complex was measured and compared to metformin HCl, a 1:1 (mol/mol) mixture of metformin hydrochloride:sodium lauryl sulfate and 1:1 (mol/mol) mixture of metformin hydrochloride:sodium laurate. The results are shown in Table 3.

TABLE 3
Octanol/Water Partition Coefficients

| Test Species | *LogD |
|---|---|
| Metformin HCl | −2.64 |
| 1:1, Metformin HCl: Sodium lauryl sulfate | −0.05 |
| 1:1, Metformin HCl: Sodium laurate | 0.06 |
| Metformin laurate | 0.44 |

*Log[C_{octanol}/C_{water}]

The complex had a logD of 0.44, a significant increase compared to metformin hydrochloride, indicating that the complex partitions more favorably in to octanol than does the salt form of metformin. The complex also had a higher logD compared to the physical mixtures of metformin hydrochloride in the fatty acid salts. This difference in logD further confirms that the complex of metformin-fatty acid is not a physical mixture of the two species, i.e., a simple loose ion pair, but is a tight ion pair.

While not wishing to be bound by specific understanding of mechanisms, the inventors reason as follows. When loose ion-pairs are placed in a polar solvent environment, it is assumed that polar solvent molecules will insert themselves in the space occupied by the ionic bond, thus driving apart the bound ions. A solvation shell, comprising polar solvent molecules electrostatically bonded to a free ion, may be formed around the free ion. This solvation shell then prevents the free ion from forming anything but a loose ion-pairing ionic bond with another free ion. In a situation wherein there are multiple types of counter ions present in the polar solvent, any given loose ion-pairing may be relatively susceptible to counter-ion competition.

This effect is more pronounced as the polarity, expressed as the dielectric constant of the solvent, increases. Based on Coulomb's law, the force between two ions with charges (q1) and (q2) and separated by a distance (r) in a medium of dielectric constant (e) is:

$$F = - \frac{{q}_{1} {q}_{2}}{4 π {ɛ}_{0} ɛ {r}^{2}} \qquad ( Equation 2 )$$

where ε₀ is the constant of permittivity of space. The equation shows the importance of dielectric constant (ε) on the stability of a loose ion-pair in solution. In aqueous solution that has a high dielectric constant (ε=80), the electrostatic attraction force is significantly reduced if water molecules attack the ionic bonding and separate the opposite charged ions.

Therefore, high dielectric constant solvent molecules, once present in the vicinity of the ionic bond, will attack the bond and eventually break it. The unbound ions then are free to move around in the solvent. These properties define a loose ion-pair.

Tight ion-pairs are formed differently from loose-ion pairs, and consequently posses different properties from a loose ion-pair. Tight ion-pairs are formed by reducing the number of polar solvent molecules in the bond space between two ions. This allows the ions to move tightly together, and results in a bond that is significantly stronger than a loose ion-pair bond, but is still considered an ionic bond. As disclosed more fully herein, tight ion-pairs are obtained using less polar solvents than water so as to reduce entrapment of polar solvents between the ions.

For additional discussion of loose and tight ion-pairs, D. Quintanar-Guerrero et al., Pharm. Res., 14(2):119-127 (1997).

The difference between loose and tight ion-pairing also can be observed using chromatographic methods. Using reverse phase chromatography, loose ion-pairs can be readily separated under conditions that will not separate tight ion-pairs.

Bonds according to this invention may also be made stronger by selecting the strength of the cation and anion relative to one another. For instance, in the case where the solvent is water, the cation (base) and anion (acid) can be selected to attract one another more strongly. If a weaker bond is desired, then weaker attraction may be selected. Portions of biological membranes can be modeled to a first order approximation as lipid bilayers for purposes of understanding molecular transport across such membranes. Transport across the lipid bilayer portions (as opposed to active transporters, etc.) is unfavorable for ions because of unfavorable portioning. Various researchers have proposed that charge neutralization of such ions can enhance cross-membrane transport.

In the “ion-pair” theory, ionic drug moieties are paired with transport moiety counter ions to “bury” the charge and render the resulting ion-pair more liable to move through a lipid bilayer. This approach has generated a fair amount of attention and research, especially with regards to enhancing absorption of orally administered drugs across the intestinal epithelium.

While ion-pairing has generated a lot of attention and research, it has not always generated a lot of success. For instance, ion-pairs of two antiviral compounds were found not to result in increased absorption due to the effects of the ion-pair on trans-cellular transport, but rather to an effect on monolayer integrity (J. Van Gelder et al., Int. J. of Pharmaceutics, 186:127-136 (1999). The authors concluded that the formation of ion pairs may not be very efficient as a strategy to enhance trans-epithelial transport of charged hydrophilic compounds as competition by other ions found in in vivo systems may abolish the beneficial effect of counter-ions. Other authors have noted that absorption experiments with ion-pairs have not always pointed at clear-cut mechanisms (D. Quintanar-Guerrero et al., Pharm. Res., 14(2):119-127 (1997)).

The inventors have unexpectedly discovered that a problem with these ion-pair absorption experiments is that they were performed using loose-ion pairs, rather than tight ion-pairs. Indeed, many ion-pair absorption experiments disclosed in the art do not even expressly differentiate between loose ion-pairs and tight ion-pairs. One of skill has to distinguish that loose ion-pairs are disclosed by actually reviewing the disclosed methods of making the ion-pairs and noting that such disclosed methods of making are directed to loose ion-pairs not tight ion-pairs. Loose ion-pairs are relatively susceptible to counter-ion competition, and to solvent-mediated (e.g. water-mediated) cleavage of the ionic bonds that bind loose ion-pairs. Accordingly, when the drug moiety of the ion-pair arrives at an intestinal epithelial cell membrane wall, it may or may not be associated in a loose ion-pair with a transport moiety. The chances of the ion-pair existing near the membrane wall may depend more on the local concentration of the two individual ions than on the ion bond keeping the ions together. Absent the two moieties being bound when they approached an intestinal epithelial cell membrane wall, the rate of absorption of the non-complexed drug moiety might be unaffected by the non-complexed transport moiety. Therefore, loose ion-pairs might have only a limited impact on absorption compared to administration of the drug moiety alone.

In contrast, the inventive complexes possess bonds that are more stable in the presence of polar solvents such as water. Accordingly, the inventors reasoned that, by forming a complex, the drug moiety and the transport moiety would be more likely to be associated as ion-pairs at the time that the moieties would be near the membrane wall. This association would increase the chances that the charges of the moieties would be buried and render the resulting ion-pair more liable to move through the cell membrane.

In an embodiment, the complex comprises a tight ion-pair bond between the drug moiety and the transport moiety. As discussed herein, tight ion-pair bonds are more stable than loose ion-pair bonds, thus increasing the likelihood that the drug moiety and the transport moiety would be associated as ion-pairs at the time that the moieties would be near the membrane wall. This association would increase the chances that the charges of the moieties would be buried and render the tight ion-pair bound complex more liable to move through the cell membrane.

It should be noted that the inventive complexes may improve absorption relative to the non-complexed drug moiety throughout the G.I. tract, not just the lower G.I. tract, as the complex is intended to improve transcellular transport generally, not just in the lower G.I. tract. For instance, if the drug moiety is a substrate for an active transporter found primarily in the upper G.I., the complex formed from the drug moiety may still be a substrate for that transporter. Accordingly, the total transport may be a sum of the transport flux effected by the transporter plus the improved transcellular transport provided by the present invention. In an embodiment, the inventive complex provides improved absorption in the upper G.I. tract, the lower G.I. tract, and both the upper G.I. tract and the lower G.I. tract.

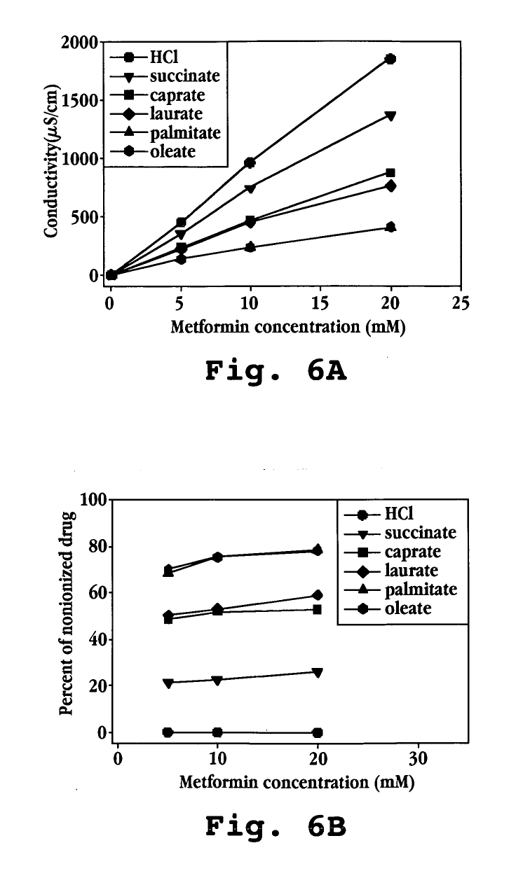

In a study conducted in support of the invention, metformin-fatty acid complexes were prepared according to the procedure described in Example 1 using the fatty acids capric acid, lauric acid, palmitic acid, and oleic acid. A complex of metformin and ethylene succinic acid was also prepared. The complexes were characterized by melting points and solubility and the data is summarized in Table 4A. Additionally, the conductivity of the various complexes in aqueous solutions (pH≅5.8) was measured with a CDM 83 conductivity meter (Radiometer Copenhagen) at 23° C. The values are summarized in Table 4B and presented graphically in FIG. 6A.

TABLE 4A

| Metformin Salt or | Melting Point | H₂O Solubility |
|---|---|---|
| Complex | (° C.) | (4 h) |
| HCl | 238 | >300 |
| succinate | 243 | 95 |
| palmitate | 150 | 12 |
| Oleate | 138 | 53 |
| Caprate | 153 | gelation |
| Laurate | 151 | gelation |

TABLE 4B

| | Conductivity (μS/cm) | | | | | |
|---|---|---|---|---|---|---|
| Met- | | | | | | |
| formin | Met- | Met- | Met- | Met- | Met- | Met- |
| Concen- | formin | formin | formin | formin | formin | formin |
| trate | HCl | succinate | caprate | laurate | palmitate | oleate |
| 20 (mM) | 1850 | 1370 | 872 | 758 | 398 | 405 |
| 10 (mM) | 958 | 741 | 461 | 450 | 237 | 235 |
| 5 (mM) | 452 | 355 | 233 | 225 | 144 | 136 |
| 0 (mM) | 1.26 | 1.26 | 1.26 | 1.26 | 1.26 | 1.26 |

FIG. 6A shows the conductivity, in microsiemens/centimeter (μS/cm) as a function of metformin concentration for metformin HCl (circles), metformin complexed with succinate (inverted triangles), caprate (squares), laurate (diamonds), palmitate (triangles), and oleate (octagons). Metformin HCl had the highest conductivity at all concentrations. The complexes had a lower conductivity than metformin hydrochloride, with a decreasing conductivity with increasing fatty acid carbon number apparent.

On the assumption that the conductivity (k) is proportional to the concentration of charged ions and that metformin HCl is 100% charged, the percentage of nonionized drug (f) was estimated by the following equation. It was also assumed that diffusion effects of the varying sizes of the fatty acid molecules was negligible.
f=(1−k/k_{HCL})×100 (Equation 3)

FIG. 6B shows the percent of non-ionized drug for each of the complexes as a function of metformin concentration, determined from Equation 3. Metformin HCl (circles) is completely ionized, whereas metformin-succinate (inverted triangles) is about 80% ionized. The complexes metformin-caprate (squares) and metformin-laurate (diamonds) and about 50% ionized, and metformin-palmitate (triangles), and metformin-oleate (octagons) are about 30% ionized. Again this data establishes a difference between the ion pair metformin hydrochloride and the metformin-fatty acid complexes.

$$f = ( 1 - \frac{k}{{k}_{HCl}} ) \times 100$$

Defining a dissociation factor as one hundred minus the percent of nonionized drug (f), in one embodiment, the complex of the present invention exhibit a dissociation factor of between 5 to 90, more preferably 5 to 85, more preferably 10 to 70, and even more preferably 20to 65 in a pH 5.8 aqueous environment at concentrations of 20 millimoles of metformin per liter.

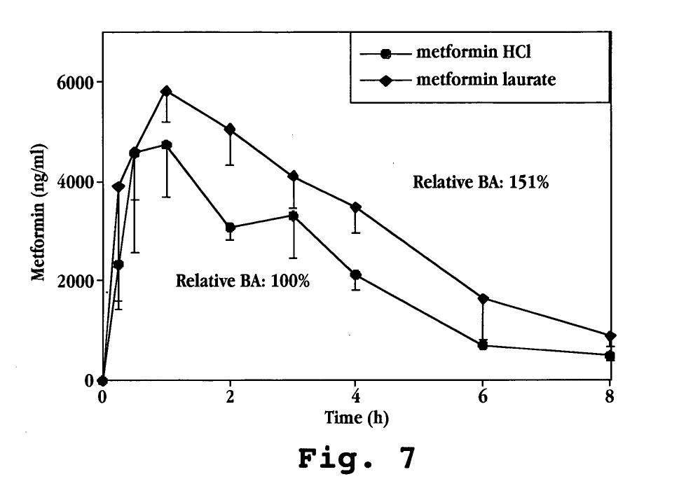

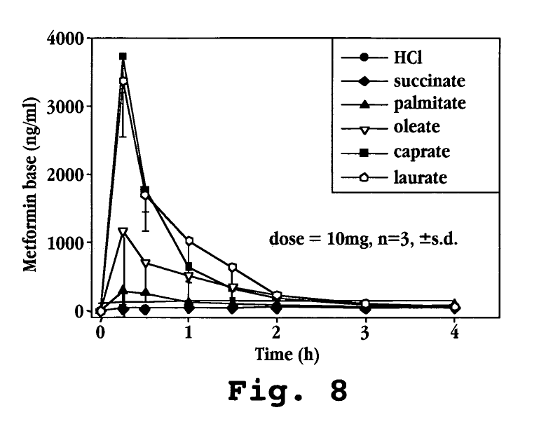

The colonic absorption of the metformin-laurate complex was characterized in vivo using an oral gavage rat model. As described in Example 2, fasted rats were treated with 40 mg/rat of metformin hydrochloride or the metformin-laurte complex. Blood samples were drawn for analysis of metformin concentration, and the results are shown in FIG. 7. The plasma concentration in rats given metformin HCl (circles) by oral gavage reached a plasma concentration maximum about 1 hour after treatment, with a Cmax of about 4080 ng/mL. Rats treated by oral gavage with the metformin-laurate complex (diamonds) had a plasma concentration maximum about 1 hour after treatment, with a Cmax of about 5090 ng/mL. The plasma concentration for rats treated with the complex was higher at all test points in the period 1-8 hours after treatment. Analysis of the data showed that the relative bioavailability of metformin when administered in the form of the complex was 151%, relative to the bioavailability of metformin when administered intravenously as metformin HCl (100% bioavailability).

The in vivo colonic absorption of the complexes was also evaluated using a flush ligated colonic model in rats. As described in Example 3, a 10 mg/rat dose of various complexes was intubated into the ligated colon of rats. The rats (n=3) in each test group were dosed with metformin HCl, metformin succinate dimer, metformin palmitate, metformin oleate, metformin caprate, or metformin laurate. Another group of rats was given 1 mg of metformin HCl intravenously. Blood samples were withdrawn periodically for analysis of metformin base concentration in the blood. The data is shown in FIG. 8.

FIG. 8 shows the metformin plasma concentration, in ng/mL, in rats as a function of time, in hours, for metformin HCl (circles), metformin complexed with succinate (diamonds), palmitate (triangles), oleate (inverted triangles), caprate (squares), and laurate (octagons). The highest blood plasma concentrations were obtained from the complexes prepared from lauric acid (circles) and with capric acid (squares). Complexes with palmitic acid (triangles) and oleic acid (inverted triangles) achieved metformin plasma concentrations lower than that achieved form the complexes with lauric acid and capric acid, but higher than the plasma concentration provided by metformin HCl or by metformin succinate.

Table 5 shows the relative Cmax (maximum plasma concentration of metformin base for each complex relative to the plasma concentration of metformin HCl), and the relative bioavailability of each complex normalized to the bioavailability of metformin HCl given via intubation to a ligated (fourth column) and relative to the bioavailability of metformin HCl given intravenously (third column).

TABLE 5

| Metformin | | Area Under | Bioavailability | Bioavailability |
|---|---|---|---|---|
| Test | | the Curve | Relative to IV | Relative to IV |
| Com- | Relative | (0-4 hrs, based | Dose¹ | Dose² |
| pound | Cₘₐₓ | on 1 mg base/rat) | (%) | (Fold-Increase) |
| HCl (i.v.) | 1.0 | 692.5 | 100 | — |
| HCl | | 29.2 | 4.2 | 1 |
| succinate | 0.6 | 21.4 | 3.1 | 0.7 |
| palmitate | 3.6 | 144.3 | 20.8 | 5 |
| Oleate | 14.1 | 408.3 | 59.0 | 14 |
| Caprate | 45.0 | 548.0 | 79.1 | 19 |
| Laurate | 40.8 | 674.5 | 97.4 | 23 |

¹AUC achieved by each complex normalized to the AUC of metformin HCl given intravenously; (ng · h/mL-mg).
²AUC achieved by each complex normalized to the AUC of metformin HCl given via intubation to ligated colon.

Metformin when provided for absorption to the colon in the form of a metformin-transport moiety complex is significantly enhanced, as seen by the nearly 5-fold increase in bioavailability achieved with a metformin-palmitate complex, relative to that of the HCl salt. The oleate complex yielded a 14-fold improvement in bioavailability relative to that of the HCl salt. The metformin-caprate complex provided an almost 18-fold improvement bioavailability relative to that of the HCl salt. The metformin-laurate complex yielded a greater than 20-fold improvement in bioavailability relative to that of the HCl salt. Accordingly, the invention contemplates a compound comprised of, consisting essentially of, or consisting of a complex formed of metformin and a transport moiety, wherein the complex provides at least a 5 fold increase, more preferably at least a 15 fold increase, and more preferably at least a 20 fold increase in colonic absorption relative to colonic absorption of metformin HCl, as evidenced by metformin bioavailability determined from metformin plasma concentration. Thus, metformin when administered in the form of a metformin-transport moiety complex provides a significantly enhanced colonic absorption of metformin into the blood.

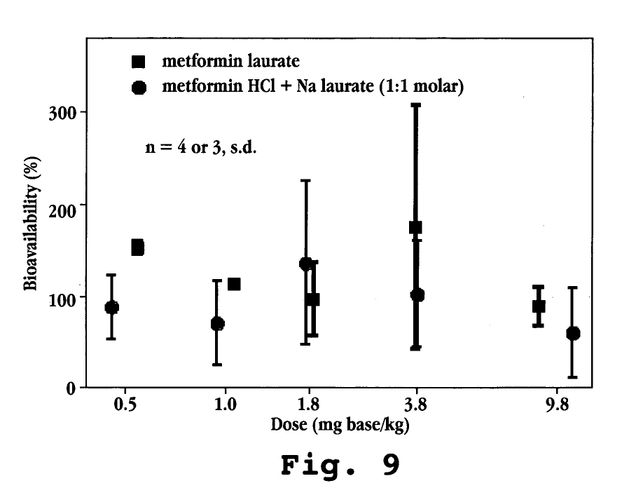

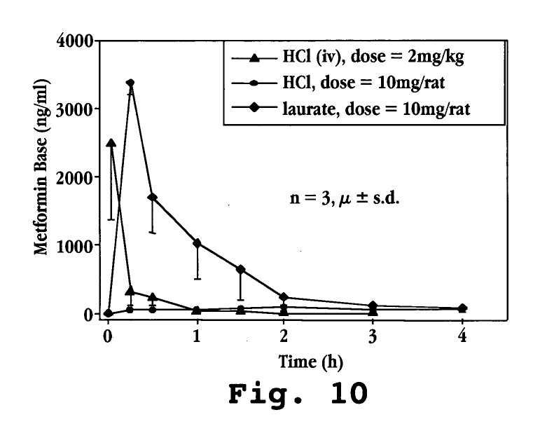

Another study was conducted using the flush-ligated colonic model described in Example 3 to compare the bioavailability of metformin when provided in the form of a complex to the bioavailability of metformin when provided as a physical mixture of metformin HCl and sodium laurate (1:1 molar ratio). Various doses of the two test formulations (metformin-laurate complex and 1:1 molar ratio metformin HCl:sodium laurate) or of metformin HCl were intubated into the ligated colon. Plasma samples were analyzed for metformin concentration and bioavailability determined, relative to the bioavailability of intravenously administered metformin. The results are shown in FIG. 9.

FIG. 9 shows the percent bioavailability as a function of metformin dose, in mg base/kg, of the physical mixture of metformin HCl and sodium laurate (circles) and of the metformin laurate complex (squares). The complex had a higher bioavailability with lower variability than the physical mixture.

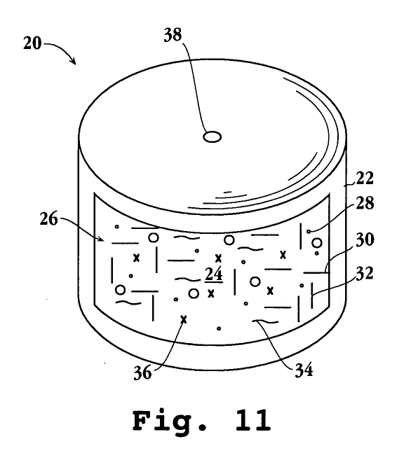

FIG. 10 shows the data from Tables A, F, and G in Example 3, to illustrate the pharmacokinetics of the complex (diamonds) compared to metformin HCl administered via intubation to the ligated colon (circles) or intravenously (triangles). The complex provides a higher colonic absorption than the salt form of the drug, and has a longer lasting blood concentration that intravenous administration.

III. Exemplary Dosage Forms and Methods of Use

The complex described above provides an enhanced absorption rate in the G.I. tract, and in particular in the lower G.I. tract. Dosage forms and methods of treatment using the complex and its increased colonic absorption will now be described. It will be appreciated that the dosage forms described below are merely exemplary.

A variety of dosage forms are suitable for use with the metformin-transport moiety complex. As discussed above, a dosage form that provides once daily dosing to achieve a therapeutic efficacy for at least about 15 hours, more preferably for at least 18 hours, and still more preferably for at least about 20 hours. The dosage form may be configured and formulated according to any design that delivers a desired dose of metformin. Typically, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Remington's Pharmaceutical Sciences, 18^{th} Ed., pp. 1682-1685 (1990).

A specific example of a dosage form suitable for use with the metformin-transport moiety complex is an osmotic dosage form. Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. An advantage to osmotic systems is that their operation is pH-independent and, thus, continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35:1-21 (1995). Osmotic dosage forms are also described in detail in the following U.S. patents, each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

An exemplary dosage form, referred to in the art as an elementary osmotic pump dosage form, is shown in FIG. 11. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising a metformin-transport moiety complex 28 in an admixture with selected excipients. The excipients are adapted to provide an osmotic activity gradient for attracting fluid from an external environment through wall 22 and for forming a deliverable metformin-transport moiety complex formulation upon imhibition of fluid. The excipients may include a suitable suspending agent, also referred to herein as drug carrier 30, a binder 32, a lubricant 34, and an osmotically active agent referred to as an osmagent 36. Exemplary materials for each of these components are provided below.

Semi-permeable wall 22 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall include homopolymers and copolymers, such as, cellulose esters, cellulose ethers, and cellulose ester-ethers. Flux-regulating agents can be admixed with the wall-forming material to modulate the fluid permeability of the wall. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across wall 22 due to the presence of osmotically-active agents causes gastric fluid to be imbibed through the wall, swelling of the drug layer, and formation of a deliverable metformin-transport moiety formulation (e.g., a solution, suspension, slurry or other flowable composition) within the internal compartment. The deliverable metformin-transport moiety formulation is released through an exit 38 as fluid continues to enter the internal compartment. Even as drug formulation is released from the dosage form, fluid continues to be drawn into the internal compartment, thereby driving continued release. In this manner, metformin-transport moiety is released in a sustained and continuous manner over an extended time period.

Preparation of a dosage form like that shown in FIG. 11 is described in Example 4.

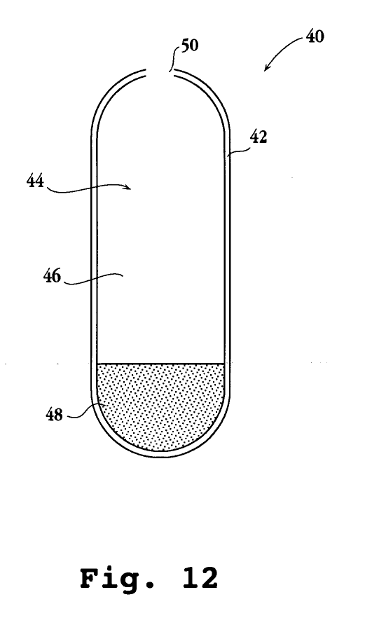

FIG. 12 is a schematic illustration of another exemplary osmotic dosage form. This dosage form is described in detail in U.S. Pat. Nos.: 4,612,008; 5,082,668; and 5,091,190, which are incorporated by reference herein. In brief, dosage form 40, shown in cross-section, has a semi-permeable wall 42 defining an internal compartment 44. Internal compartment 44 contains a bilayered-compressed core having a drug layer 46 and a push layer 48. As will be described below, push layer 48 is a displacement composition that is positioned within the dosage form such that as the push layer expands during use, the materials forming the drug layer are expelled from the dosage form via one or more exit ports, such as exit port 50. The push layer can be positioned in contacting layered arrangement with the drug layer, as illustrated in FIG. 12, or can have one or more intervening layers separating the push layer and drug layer.

Drug layer 46 comprises a metformin-transport moiety complex in an admixture with selected excipients, such as those discussed above with reference to FIG. 11. An exemplary dosage form can have a drug layer comprised of metformin-laurate complex, a poly(ethylene oxide) as a carrier, sodium chloride as an osmagent, hydroxypropylmethylcellulose as a binder, and magnesium stearate as a lubricant.

Push layer 48 comprises osmotically active component(s), such as one or more polymers that imbibes an aqueous or biological fluid and swells, referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the semi-permeable wall, the osmopolymer(s) swell and push against the drug layer to cause release of the drug from the dosage form via the exit port(s).

The push layer can also include a component referred to as a binder, which is typically a cellulose or vinyl polymer, such as poly-n-vinylamide, poly-n-vinylacetamide, poly(vinyl pyrrolidone), poly-n-vinylcaprolactone, poly-n-vinyl-5-methyl-2-pyrrolidone, and the like. The push layer can also include a lubricant, such as sodium stearate or magnesium stearate, and an antioxidant to inhibit the oxidation of ingredients. Representative antioxidants include, but are not limited to, ascorbic acid, ascorbyl palmitate, butylated hydroxyanisole, a mixture of 2 and 3 tertiary-butyl-4-hydroxyanisole, and butylated hydroxytoluene.

An osmagent may also be incorporated into the drug layer and/or the push layer of the osmotic dosage form. Presence of the osmagent establishes an osmotic activity gradient across the semi-permeable wall. Exemplary osmagents include salts, such as sodium chloride, potassium chloride, lithium chloride, etc. and sugars, such as raffinose, sucrose, glucose, lactose, and carbohydrates.

With continuing reference to FIG. 12, the dosage form can optionally include an overcoat (not shown) for color coding the dosage forms according to dose or for providing an immediate release of metformin or another drug.

In use, water flows across the wall and into the push layer and the drug layer. The push layer imbibes fluid and begins to swell and, consequently, pushes on drug layer 44 causing the material in the layer to be expelled through the exit orifice and into the gastrointestinal tract. Push layer 48 is designed to imbibe fluid and continue swelling, thus continually expelling drug from the drug layer throughout the period during which the dosage form is in the gastrointestinal tract. In this way, the dosage form provides a continuous supply of metformin-transport moiety complex to the gastrointestinal tract for a period of 15 to 20 hours, or through substantially the entire period of the dosage form's passage through the G.I. tract. Since metformin-transport moiety complex is readily absorbed in both the upper and lower G.I. tracts administration of the dosage form provides delivery of metformin into the blood stream over the 15-20 hour period of dosage form transit in the G.I. tract.

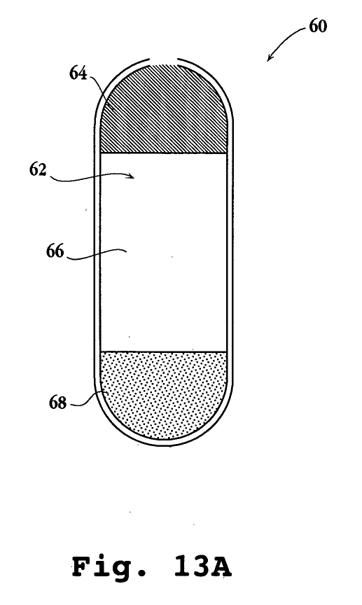

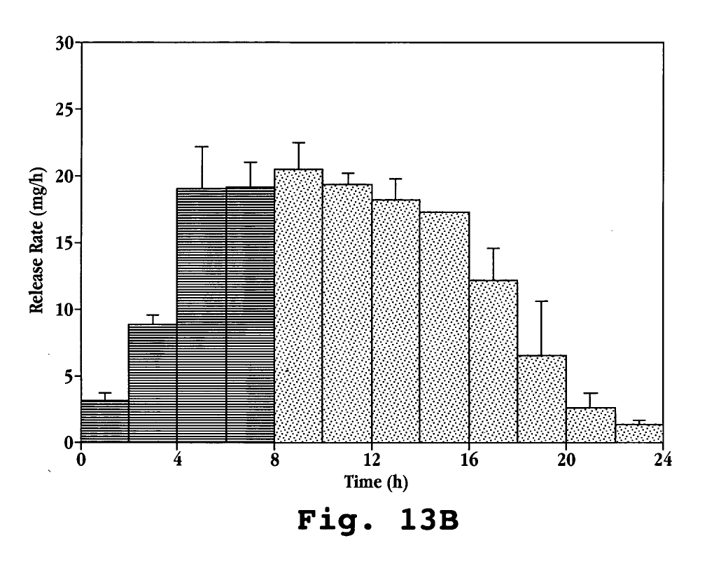

Another exemplary dosage form is shown in FIG. 13A. Osmotic dosage form 60 has a tri-layered core 62 comprised of a first layer 64 of metformin HCl, a second layer 66 of a metformin-transport moiety complex, and a third layer 68 referred to as a push layer. Dosage forms of this type are described in detail in U.S. Pat. Nos.: 5,545,413; 5,858,407; 6,368,626, and 5,236,689, which are incorporated by reference herein. As set forth in Example 5, tri-layered dosage forms were prepared to have a first layer of 85.0 wt % metformin hydrochloride, 10.0 wt % polyethylene oxide of 100,000 molecular weight, 4.5 wt % polyvinylpyrrolidone having a molecular weight of about 35,000 to 40,000, and 0.5 wt % magnesium stearate. The second layer was comprised 93.0 wt % metformin-laurate complex (prepared as described in Example 1), 5.0 wt % polyethylene oxide 5,000,000 molecular weight, 1.0 wt % polyvinylpyrrolidone having molecular weight of about 35,000 to 40,000 and 1.0 wt % magnesium stearate.

The push layer consisted of 63.67 wt % of polyethylene oxide, 30.00 wt % sodium chloride, 1.00 wt % ferric oxide, 5.00 wt % hydroxypropylmethylcellulose, 0.08 wt % butylated hydroxytoluene and 0.25 wt % magnesium stearate. The semi-permeable wall was comprised of 80.0 wt % cellulose acetate having a 39.8 % acetyl content and 20.0 % polyoxyethylene-polyoxypropylene copolymer.

The dissolution rate of metformin from the dosage form shown in FIG. 13A was determined according to procedure set forth in Example 5. The results are shown in FIG. 13B, where the release rate of metformin, in mg/hour, is shown as a function of time, in hours. Four hours after contact with an aqueous environment, the dosage form begins to release a nearly uniform amount of drug for the subsequent 12 hours, with release of drug beginning to decrease at times greater than 16 hours after contact with an aqueous environment. Release of metformin hydrochloride, present in the drug layer adjacent the exit orifice, is released initially. About 8 hours after contact with an aqueous environment, release of metformin-transport moiety complex occurs, and continues at a substantially constant rate for 8 hours longer. It will be appreciated that this dosage form is designed to release metformin hydrochloride while in transit in the upper G.I. tract, corresponding approximately to the first eight hours of transit, as indicated by the dashed bars. Metformin-transport moiety complex is released as the dosage form travels through the lower G.I. tract, approximately corresponding to times longer than about 8 hours after ingestion, as indicated by the dotted bars in FIG. 13B. This design takes advantage of the increased colonic absorption provided by the complex.

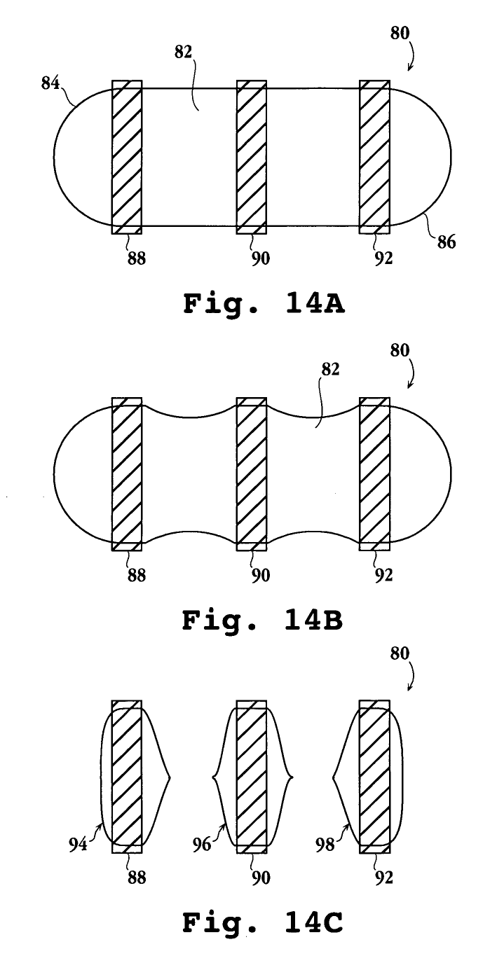

FIGS. 14A-14C illustrate another exemplary dosage form, known in the art and described in U.S. Pat. Nos. 5,534,263; 5,667,804; and 6,020,000, which are specifically incorporated by reference herein. Briefly, a cross-sectional view of a dosage form 80 is shown prior to ingestion into the gastrointestinal tract in FIG. 14A. The dosage form is comprised of a cylindrically shaped matrix 82 comprising a metformin-transport moiety complex. Ends 84, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above and in Example 6 below.

After ingestion of dosage form 80, regions of matrix 82 between bands 88, 90, 92 begin to erode, as illustrated in FIG. 14B. Erosion of the matrix initiates release of the metformin-transport moiety complex into the fluidic environment of the G.I. tract. As the dosage form continues transit through the G.I. tract, the matrix continues to erode, as illustrated in FIG. 14C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

It will be appreciated the osmotic dosage forms described in FIGS. 11-14 are merely exemplary of a variety of dosage forms designed for and capable of achieving delivery of a metformin-transport moiety complex to the lower G.I. tract. Those of skill in the pharmaceutical arts can identify other dosage forms that would be suitable.

In another aspect, the invention provides a method for treating hyperglycemia in a subject by administering a composition or a dosage form that contains a complex of metformin and a transport moiety, the complex characterized by a hybrid bond or a tight ion pair bond between the metformin and the transport moiety. The method finds use in treating persons with non-insulin-dependent diabetes mellitus (Type II diabetes) and/or insulin-dependent diabetes mellitus (Type I diabetes). A composition comprising the complex and a pharmaceutically-acceptable vehicle are administered to the patient, typically via oral administration.

The dose administered is generally adjusted in accord with the age, weight, and condition of the patient, taking into consideration the dosage form and the desired result. In general, the dosage forms and compositions of the metformin-transport moiety complex are administered in amounts recommended for metformin HCl (Glucophage®, Bristol-Myers Squibb Co.) as set forth in the Physician's Desk Reference. For example, oral dosage of metformin HCl is individualized on the basis of effectiveness and tolerance, while not exceeding the maximum daily recommended dose of 2550 mg in adults and 2000 mg in pediatric patients. Metformin HCl is typically administered in divided doses with meals and is often initiated at a low dose, typically of about 850 mg/day, with gradual escalation to permit identification of a minimum therapeutically effective amount required for an individual's anti-hyperglycemic activity. Thus, in one embodiment, an dosage form that provides a daily metformin dose of between 500-2550 mg is provided, where the metformin is provided in the form of a metformin-transport moiety complex.

In another aspect, the invention contemplates administering a metformin-transport moiety complex in combination with a second therapeutic agent, for treatment of hyperglycemia and for management of weight, particularly in Type II diabetic subjects. Preferred second therapeutic agents are those useful in the treatment of obesity, diabetes mellitus, especially Type II diabetes, and conditions associated with diabetes mellitus.

Exemplary second therapeutic agents include, but are not limited to, an compounds classified as an alpha glucosidase inhibitor, a biguanide (other than metformin), an insulin secretagogue, an antidiabetic agent, or an insulin sensitizer. Exemplary alpha glucosidase inhibitors include acarbose, emiglitate, miglitol, voglibose. A suitable antidiabetic agent is insulin. Biguanides include buformin and phenformin. Suitable insulin secretagogues include sulphonylureas, such as glibenclamie, glipizide, gliclazide, glimepiride, tolazamide, tolbutamine, acetohexamide, carbutamide, chlorpropamide, glibornuride, gliquidone, glisentide, glisolamide, glisoxepide, glyclopyamide, repaglinide, nateglinide, and glycyclamide. Insulin sensitizers include PPAR-gamma agonist insulin sensitizers (see WO97/31907), such as 2-(1-carboxy-2-{4-2-(5-methyl-2-phenyl-oxazol-4-yl)-ethoxy]-phenyl-ethylamino)-benzoic acid methyl ester and 2(S)-(2-benzoyl-phenylamino)-3-4-[2-(5-methyl-2-phenyl-oxazol-4-yl)-ethoxy]-phenyl}-propionic acid.

The second therapeutic agent is preferably an anti-diabetic compound, such as insulin signaling pathway modulators, like inhibitors of protein tyrosine phosphatases (PTPases), non-small molecule mimetic compounds and inhibitors of glutamine-fructose-6-phosphate amidotransferase (G FAT), compounds influencing a dysregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, or the pharmaceutically acceptable salts of such a compound and optionally at least one pharmaceutically acceptable carrier; for simultaneous, separate or sequential use, particularly in the prevention, delay of progression or treatment of conditions mediated by DPP-IV, in particular conditions of impaired glucose tolerance (IGT), conditions of impaired fasting plasma glucose, metabolic acidosis, ketosis, arthritis, obesity and osteoporosis, and preferably diabetes, especially type 2 diabetes mellitus. Such a combination is preferably a combined preparation or a pharmaceutical composition.

In a combined treatment method, the metformin-transport moiety complex and the second therapeutic agent are administered simultaneously or sequentially, by the same or different routes of administration.

In a preferred embodiment, the second therapeutic agent is a dipeptidyl peptidase IV (DPP-IV) inhibitor. Dipeptidyl peptidase IV is a post-proline/alanine cleaving serine protease found in various tissues in the body, including kidney, liver, and intestine. The protease removes the two N-terminal amino acids from proteins having proline or alanine in the position 2. DPP-IV can be used in the control of glucose metabolism because its substrates include the insulinotropic hormones glucagons like peptide-1 (GLP-1) and gastric inhibitory peptide (GIP). GLP-1 and GIP are active only in their intact forms; removal of their two N-terminal amino acids inactivates them (Holst, J. et al., Diabetes, 47:1663 (1998)).

Thus, inhibitors of DPP-IV have been described, for example, in U.S. Pat. Nos. 6,124,305; 6,107,317; and in PCT Publication Nos. WO99/61431; WO98/19998; WO95/15309; WO98/18736. The inhibitors can be peptidic or non-peptidic, such as 1[2-(5-cyanopyridin-2yl)aminoethylamino]acetyl-2-cyano-(S)-pyrrolidine and (2S)-1-[(2S)-2-amino-3,3-dimethylbutanoyl]-2-pyrrolidinecarbonitrile.

A method for treating a subject having Type II diabetes is contemplated, where the subject is treated with a DPP-IV inhibitor in combination with a metformin-transport moiety complex. The combined agents produces a greater beneficial effect than achieved for either agent alone or for a combination of a DPP-IV inhibitor and metformin not in complex form. The metformin-transport moiety complex is preferably administered orally in a once-daily dosage form, to take full advantage of the enhanced colonic absorption provided by the complex. The DPP-IV inhibitor can be administered by any route suitable for the compound and the patient.

In one embodiment, the combined treatment regimen is for use in reducing or preventing body weight gain in overweight or obese patients with Type II diabetes. It has been recently shown that a combination therapy of metformin with DPP-IV inhibitor leads to reduced food intake and body weight gain in Zucker fa/fa rats (Yasuda, N. et al., J. Pharmacol. Experimental Therap., 310(2):614 (2004)). The invention provides an improved combination regimen by administering metformin as a metformin-transport moiety complex to achieve an enhanced colonic absorption.

From the foregoing, it can be seen how various objects and features of the invention are met. A complex consisting of metformin and a transport moiety, the metformin and transport moiety associated by a hybrid bond or by a tight-ion pair bond, provides an enhanced colonic absorption of metformin, relative to that observed for metformin HCl. The complex is prepared from a novel process, where metformin in base form is contacted with a transport moiety solubilized in an organic solvent, the organic solvent being less polar than water, the lower polarity evidenced, for example, by a lower dielectric constant. Contact of metformin base with the transport moiety-solvent mixture results in formation of a complex between metformin and the transport moiety, where the two species are associated by a bond that is not an ionic bond and that is not a covalent bond, but is hybrid bond or a tight-ion pair bond.

### IV. EXAMPLES

The following examples further illustrate the invention described herein and are in no way intended to limit the scope of the invention.

Methods

1. HPLC: Reverse phase was conducted on a Hewlett Packard 1100 liquid chromatograph with an evaporative light scattering detector and using a C3 column (Agilent Zorbax SB C3, 5 μm, 3.0×75 mm. A mobile phase of water:acetonitrile 50:50 v:v was used. Column temperature was 40° C. and the flow rate was 0.5 mL/min.

### Example 1

### Preparation of Metformin-Transport Moiety Complex

Materials:

| | |
|---|---|
| metformin hydrochloride | 13.0 g |
| lauric acid | 16.0 g |
| methanol | 675 mL |
| acetone | 300 mL |
| demineralized water | 14 mL |
| anionic resin (Amberlyst A-26 (OH)) | 108 g |

Preparation of Metformin Base
1. The ion exchange column was packed with the anionic resin, Amberlyst A-26 (OH) and a net weight was obtained.
2. The column was rinsed first with deionized (DI) water (backflush) and then rinsed with methanol containing 2% v/v DI water, with care taken to not allow the column to dry out.
3. Metformin hydrochloride was dissolved in an eluant comprised of 365 mL methanol containing 2% DI water by volume.
4. The solution of step 3 was passed through the column dropwise using a separatory funnel and the eluate collected. The total metformin hydrochloride passed through was calculated to be less than the ion exchange resin's equilibrating point (capacity). The column was rinsed with approximately an equal volume of eluant. A total of 690 mL of eluate of the metformin base was collected.
5. The combined eluates were evaporated to dryness under vacuo at an external temperature of 40° C., raised to 65° C. at the end of the concentration step to remove all the remaining water. This concentration step was carried out in the most expeditious manner due to the instability of the metformin base.

Complex Formation
6. A lauric acid-acetone solution, 16.0 g lauric acid dissolved in 300 mL acetone, was prepared. The concentrated metformin base from step 5 was dissolved using several washings of acetone and these washings were immediately filtered in the presence of filter-aid to remove any unconverted metformin hydrochloride. The filtrate was collected in an Erlenmeyer flask and, with stirring, the lauric acid-acetone solution was added at a fast drop, using a sepratory funnel. Metformin laurate precipitated out. Stirring was continued overnight at ambient temperature (20-25° C.).
7. The mixture of solvent and precipitated metformin laurate was filtered through a Buchner funnel. The filter cake was rinsed with 4×200 mL acetone and then dried under vacuum suction for an hour. The filter cake was scraped off the filter paper and weighed. The melting point was determined in a capillary tube. Final drying was in a vacuum oven for 3 hours at ambient temperature was done.

The procedure resulted in formation of a complex of metformin laurate with a melting point of 150°-153° C. The melting point of metformin hydrochloride is reported as 225° C. Total Yield=75% relative to theoretical amount calculated from the stoicheometric amounts of metformin hydrochloride and lauric acid used.

### Example 2

### In Vivo Colonic Absorption Using Oral Gavage Rat Model

Eight rats were randomized into two treatment groups. After being fasted for 12-24 hours, the first group was given by oral gavage 40 mg/kg free base equivalent of metformin hydrochloride. The second group received by oral gavage 40 mg/kg free base equivalent of metformin laurate complex, prepared as described in Example 1. Blood samples were taken from the tail vein 15 minutes, 30 minutes, 1 hour, 1.5 hours, 2 hours, 3 hours, 4 hours, 6 hours, and 8 hours after oral gavage. The metformin plasma concentration was analyzed by LC/MS/MS. The results are shown in FIG. 7.

At the end of the study, the rats were executed and a macroscopic evaluation of the G.I. tract of the test animals was conducted to look for signs of irritation. No irritation in the rats treated with the complex or with metformin HCl was observed.

### Example 3

### In Vivo Colonic Absorption Using Flushed Ligated Colonic Model in Rats

An animal model commonly known as the “intracolonic ligated model” was employed. Fasted, 0.3-0.5 kg Sprague-Dawley male rats were anesthetized and a segment of proximal colon was isolated. The colon was flushed of fecal materials. The segment was ligated at both ends while a catheter was placed in the lumen and exteriorized above the skin for delivery of test formulation. The colonic contents were flushed out and the colon was returned to the abdomen of the animal. Depending on the experimental set up, the test formulation was added after the segment was filled with 1 mL/kg of 20 mM sodium phosphate buffer, pH 7.4, to more accurately simulate the actual colon environment in a clinical situation.

Rats were allowed to equilibrate for approximately 1 hour after surgical preparation and prior to exposure to each test formulation. Metformin HCl or a metformin-fatty acid complex were administered as an intracolonic bolus at dosages of 10 mg metformin HCl/rat or 10 mg metformin complex/rat. Rats were treated with metformin-fatty acid complexes prepared as described in Example 1, with the fatty acids capric acid, lauric acid, palmitic acid, and oleic acid, and with a succinate acid dimer. Blood samples were obtained from the jugular catheter at 0, 15, 30, 60, 90, 120, 180 and 240 minutes after administration of the test formulation and analyzed for blood metformin concentration. Tables A-F below show for each complex and for each rat the concentration of metformin base detected in the blood plasma measured in nanograms per millileter at each time point.

TABLE A
Metformin HCl

| | Rat1 | Rat2 | Rat3 | | Standard |
|---|---|---|---|---|---|
| Time (h) | (ng/mL) | (ng/mL) | (ng/mL) | Average | Deviation |
| 0 | 0 | 0 | 0 | 0.0 | 0.0 |
| 0.25 | 48.9 | 30.7 | 52.3 | 44.0 | 11.6 |
| 0.5 | 48.4 | 22.2 | 61.2 | 43.9 | 19.9 |
| 1 | 16.5 | 56.6 | 67.5 | 46.9 | 26.9 |
| 1.5 | 14 | 79 | 99 | 64.0 | 44.4 |
| 2 | 27.3 | 124 | 96.6 | 82.6 | 49.8 |
| 3 | 28.6 | | 81 | 54.8 | 37.1 |
| 4 | 28.2 | | 83.9 | 56.1 | 39.4 |

TABLE B
Metformin Succinate

| | Rat1 | Rat2 | Rat3 | | Standard |
|---|---|---|---|---|---|
| Time (h) | (ng/mL) | (ng/mL) | (ng/mL) | Average | Deviation |
| 0 | 0 | 0 | 0 | 0.0 | 0.0 |
| 0.25 | 19.9 | 42.6 | 19 | 27.2 | 13.4 |
| 0.5 | 19.5 | 53.6 | 18.9 | 30.7 | 19.9 |
| 1 | 24.3 | 89.8 | 19 | 44.4 | 39.4 |
| 1.5 | 26.2 | 78.4 | 15.7 | 40.1 | 33.6 |
| 2 | 44.1 | 59.1 | 13.2 | 38.8 | 23.4 |
| 3 | 36.9 | 48.3 | 15.8 | 33.7 | 16.5 |
| 4 | 62.4 | 60.4 | 19 | 47.3 | 24.5 |

TABLE C
Metformin Palmitate

| | Rat1 | Rat2 | Rat3 | | Standard |
|---|---|---|---|---|---|
| Time (h) | (ng/mL) | (ng/mL) | (ng/mL) | Average | Deviation |
| 0 | 0 | 0 | 0 | 0.0 | 0.0 |
| 0.25 | 535 | 126 | 233 | 298.0 | 212.1 |
| 0.5 | 393 | 145 | 245 | 261.0 | 124.8 |
| 1 | 147 | 43.6 | 212 | 134.2 | 84.9 |
| 1.5 | 103 | 116 | 110 | 109.7 | 6.5 |
| 2 | 86.3 | 115 | 92.5 | 97.9 | 15.1 |
| 3 | 52 | 77.7 | 76.6 | 68.8 | 14.5 |
| 4 | 57.9 | 105 | 118 | 93.6 | 31.6 |

TABLE D
Metformin Oleate

| | Rat1 | Rat2 | Rat3 | | Standard |
|---|---|---|---|---|---|
| Time (h) | (ng/mL) | (ng/mL) | (ng/mL) | Average | Deviation |
| 0 | 0 | 0 | 0 | 0.0 | 0.0 |
| 0.25 | 262 | 788 | 2450 | 1166.7 | 1142.1 |
| 0.5 | 218 | 552 | 1350 | 706.7 | 581.6 |
| 1 | 79.3 | 426 | 1040 | 515.1 | 486.5 |
| 1.5 | 87.3 | 342 | 626 | 351.8 | 269.5 |
| 2 | 59.3 | 399 | 219 | 225.8 | 170.0 |
| 3 | 27.9 | 163 | 79.3 | 90.1 | 68.2 |
| 4 | 30.2 | 122 | 37.3 | 63.2 | 51.1 |

TABLE E
Metformin Caprate

| | Rat1 | Rat2 | Rat3 | | Standard |
|---|---|---|---|---|---|
| Time (h) | (ng/mL) | (ng/mL) | (ng/mL) | Average | Deviation |
| 0 | 0 | 0 | 0 | 0.0 | 0.0 |
| 0.25 | 2750 | 3390 | 5020 | 3720.0 | 1170.4 |
| 0.5 | 1850 | 1430 | 2040 | 1773.3 | 312.1 |
| 1 | 613 | 447 | 876 | 645.3 | 216.3 |
| 1.5 | 264 | 222 | 502 | 329.3 | 151.0 |
| 2 | 177 | 243 | 193 | 204.3 | 34.4 |
| 3 | 51.8 | 97.9 | 100 | 83.2 | 27.2 |
| 4 | 40.9 | 68 | 66.7 | 58.5 | 15.3 |

TABLE F
Metformin Laurate

| | Rat1 | Rat2 | Rat3 | | Standard |
|---|---|---|---|---|---|
| Time (h) | (ng/mL) | (ng/mL) | (ng/mL) | Average | Deviation |
| 0 | 0 | 0 | 0 | 0.0 | 0.0 |
| 0.25 | 3570 | 3270 | 3280 | 3373.3 | 170.4 |
| 0.5 | 2060 | 1950 | 1100 | 1703.3 | 525.4 |
| 1 | 960 | 1590 | 544 | 1031.3 | 526.6 |
| 1.5 | 494 | 1150 | 287 | 643.7 | 450.5 |
| 2 | 237 | 378 | 112 | 242.3 | 133.1 |
| 3 | 102 | 171 | 58.3 | 110.4 | 56.8 |
| 4 | 68.1 | 77.5 | 47.4 | 64.3 | 15.4 |

For comparison, metformin HCl, in a dosage of 2 mg/kg of rat body weight was injected intravenously directly into the blood stream of three test rats. Blood samples were taken periodically over a four hour period for analysis of metformin base. The results are shown in Table G.

TABLE G
Metformin HCl (iv)

| | Rat1 | Rat2 | Rat3 | | Standard |
|---|---|---|---|---|---|
| Time (h) | (ng/mL) | (ng/mL) | (ng/mL) | Average | Deviation |
| 0 | 0 | 0 | 0 | 0.0 | 0.0 |
| 0.033333 | 1430 | 2370 | 3650 | 2483.3 | 1114.3 |
| 0.25 | 133 | 545 | 276 | 318.0 | 209.2 |
| 0.5 | 262 | 331 | 94.7 | 229.2 | 121.5 |
| 1 | 35.5 | N/A | 25.2 | 30.4 | 7.3 |
| 1.5 | 0 | 66.3 | 0 | 22.1 | 38.3 |
| 2 | 0 | 0 | 0 | 0.0 | 0.0 |
| 3 | 0 | 0 | 0 | 0.0 | 0.0 |

The results in Tables A-F are shown in graphically in FIG. 8. The Cmax and relative bioavailabilities are shown in Table 5 above.

### Example 4

### Preparation of Dosage Form Comprising a Metformin-Transport Moiety Complex

A device as shown in FIG. 11 is prepared as follows. A compartment forming composition comprising, in weight percent, 92.25% metformin-transport moiety complex, 5% potassium carboxypolymethylene, 2% polyethylene oxide having a molecular weight of about 5,000,000, and 0.5% silicon dioxide are mixed together. Next, the mixture is passed through a 40 mesh stainless steel screen and then dry blended in a V-blender for 30 minutes to produce a uniform blend. Next, 0.25% magnesium stearate is passed through an 80 mesh stainless steel screen, and the blend given an additional 5 to 8 minutes blend. Then, the homogeneously dry blended powder is placed into a hopper and fed to a compartment forming press, and known amounts of the blend compressed into ⅝ inch oval shapes designed for oral use. The oval shaped precompartments are coated next in an Accela-Cota® wall forming coater with a wall forming composition comprising 91% cellulose acetate having an acetyl content of 39.8% and 9% polyethylene glycol 3350. After coating, the wall coated drug compartments are removed from the coater and transferred to a drying oven for removing the residual organic solvent used during the wall forming procedure. Next, the coated devices are transferred to a 50° C. forced air oven for drying about 12 hours. Then, passageways are formed in the wall of the device using a laser for drilling two passageways on the major axis of each face of the dispensing device.

### Example 5

### Preparation of Dosage Form Comprising a Metformin-Transport Moiety Complex

A dosage form comprising a layer of metformin HCl and a layer of metformin-laurate complex, as illustrated in FIG. 13A, was prepared as follows.

10 grams of metformin hydrochloride, 1.18 g of polyethylene oxide of 100,000 molecular weight, and 0.53 g of polyvinylpyrrolidone having molecular weight of about 38,000 were dry blended in a conventional blender for 20 minutes to yield a homogenous blend. Next, 4 mL denatured anhydrous alcohol was added slowly, with the mixer continuously blending, to the three component dry blend. The mixing was continued for another 5 to 8 minutes. The blended wet composition was passed through a 16 mesh screen and dried overnight at room temperature. Then, the dry granules were passed through a 16 mesh screen and 0.06 g of magnesium stearate were added and all the ingredients were dry blended for 5 minutes. The fresh granules were ready for formulation as the initial dosage layer in the dosage form. The granules were comprised of 85.0 wt % metformin hydrochloride, 10.0 wt % polyethylene oxide of 100,000 molecular weight, 4.5 wt % polyvinylpyrrolidone having a molecular weight of about 35,000 to 40,000, and 0.5 wt % magnesium stearate.

The metformin-laurate layer in the dosage form was prepared as follows. First, 9.30 grams of metformin laurate complex, prepared as described in Example 1, 0.50 g polyethylene oxide of 5,000,000 molecular weight, 0.10 g of polyvinylpyrrolidone having molecular weight of about 38,000 were dry blended in a conventional blender for 20 minutes to yield a homogenous blend. Next, denatured anhydrous ethanol was added slowly to the blend with continuous mixing for 5 minutes. The blended wet composition was passed through a 16 mesh screen and dried overnight at room temperature. Then, the dry granules were passed through a 16 mesh screen and 0.10 g magnesium stearate were added and all the dry ingredients were dry blended for 5 minutes. The composition was comprised of 93.0 wt % metrformin laurate, 5.0 wt % polyethylene oxide 5,000,000 molecular weight, 1.0 wt % polyvinylpyrrolidone having molecular weight of about 35,000 to 40,000 and 1.0 wt % magnesium stearate.

A push layer comprised of an osmopolymer hydrogel composition was prepared as follows. First, 58.67 g of pharmaceutically acceptable polyethylene oxide comprising a 7,000,000 molecular weight, 5 g Carbopol® 974P, 30 g sodium chloride and 1 g ferric oxide were separately screened through a 40 mesh screen. The screened ingredients were mixed with 5 g of hydroxypropylmethylcellulose of 9,200 molecular weight to produce a homogenous blend. Next, 50 mL of denatured anhydrous alcohol was added slowly to the blend with continuous mixing for 5 minutes. Then, 0.080 g of butylated hydroxytoluene was added followed by more blending. The freshly prepared granulation was passed through a 20 mesh screen and allowed to dry for 20 hours at room temperature (ambient). The dried ingredients were passed through a 20 mesh screen and 0.25 g of magnesium stearate was added and all the ingredients were blended for 5 minutes. The final composition was comprised of 58.7 wt % of polyethylene oxide, 30.0 wt % sodium chloride, 5.0 wt % Carbopol®, 5.0 wt % hydroxypropylmethylcellulose, 1.0 wt % ferric oxide, 0.25 wt % magnesium stearate, and 0.08 wt % butylated hydroxytoluene.

The tri-layer dosage form was prepared as follows. First, 118 mg of the metformin hydrochloride composition was added to a punch and die set and tamped, then 427 mg of the metformin laurate composition was added to the die set as the second layer and again tamped. Then, 272 mg of the hydrogel composition was added and the three layers compressed under a compression force of 1.0 ton (1000 kg) into a 9/32 inch (0.714 cm) diameter punch die set, forming an intimate tri-layered core (tablet).

A semipermeable wall-forming composition was prepared comprising 80.0 wt % cellulose acetate having a 39.8% acetyl content and 20.0% polyoxyethylene-polyoxypropylene copolymer having a molecular weight of 7680-9510 by dissolving the ingredients in acetone in a 80:20 wt/wt composition to make a 5.0% solids solution. Placing the solution container in a warm water bath during this step accelerated the dissolution of the components. The wall-forming composition was sprayed onto and around the tri-layerd core to provide a 93 mg thickness semi-permeable wall.

Next, a 40 mil (1.02 mm) exit orifice was laser drilled in the semipermeable walled tri-layered tablet to provide contact of the metformin layer with the exterior of the delivery device. The dosage form was dried to remove any residual solvent and water.

The in vitro dissolution rates of the dosage form was determined by placing a dosage form in the metal coil sample holders attached to a USP Type VII bath indexer in a constant temperature water bath at 37° C. Aliquots of the release media were injected into a chromatographic system to quantify the amounts of drug released into a medium simulating artificial gastric fluid (AGF) during each testing interval. Three dosage forms were tested and the average dissolution rate is shown in FIG. 13B.

### Example 6

### Preparation of Dosage Form Comprising a Metformin-Transport Moiety Complex

A dosage form as illustrated in FIGS. 14A-14C is prepared as follows. A unit dose for prolonged release of the metformin-laurate complex is prepared as follows. The desired dose of metformin in the form of metformin-laurate complex is passed through a sizing screen having 40 wires per inch. 20 grams of a hydroxypropyl methylcellulose having a hydroxypropyl content of 8 wt %, a methoxyl content of 22 wt %, and a number average molecular weight of 27,800 grams per mole are passed through a sizing screen with 100 wires per inch. The sized powders are tumble mixed for 5 minutes. Anhydrous ethanol is added to the mixture with stirring until a damp mass is formed. The damp mass is passed through a sizing screen with 20 wires per inch. The resulting damp granules are air dried overnight, and then passed again through the 20 mesh sieve. 2 grams of the tabletting lubricant, magnesium stearate, are passed through a sizing screen with 80 wires per inch. The sized magnesium stearate is blended into the dried granules to form the final granulation.

705 mg portions of the final granulation are placed in die cavities having inside diameters of 0.281 inch. The portions are compressed with deep concave punches under a pressure head of 1 ton, forming longitudinal capsule-shaped tablets.

The capsules are fed into a Tait Capsealer Machine (Tait Design and Machine Co., Manheim, Pa.) where three bands are printed onto each capsule. The material forming the bands is a mixture of 50 wt % ethylcellulose dispersion (Surelease®, Colorcon, West Point, Pa.) and 50 wt % ethyl acrylate methylmethacrylate (Eudragit® NE 30D, RohmPharma, Weiterstadt, Germany). The bands are applied as an aqueous dispersion and the excess water is driven off in a current of warm air. The diameter of the bands is 2 millimeters.

Although the invention has been described with respect to particular embodiments, it will be apparent to those skilled in the art that various changes and modifications can be made without departing from the invention.

1. A substance comprised of metformin and a transport moiety, said metformin and said transport moiety forming a complex.

2. The substance of claim 1, wherein said transport moiety, prior to complex formation, is a fatty acid of the form CH₃(CₙH₂ₙ)COOH, where n is from 4-16.

3. The substance of claim 2, wherein said fatty acid is capric acid or lauric acid.

4. A composition, comprising,
a complex comprised of metformin and a transport moiety, and
a pharmaceutically acceptable vehicle,
wherein said composition has an absorption in the lower gastrointestinal tract at least four-fold higher than metformin hydrochloride.

5. The composition of claim 4, wherein said transport moiety is a fatty acid, prior to complex formation, of the form CH₃(CₙH₂ₙ)COOH, where n is from 4-16.

6. The composition of claim 5, wherein said fatty acid is capric acid or lauric acid.

7. A dosage form comprising the composition of claim 4.

8. A dosage form comprising the substance of claim 1.

9. The dosage form of claim 8, wherein the dosage form is an osmotic dosage form.

10. The dosage form of claim 9, comprised of (i) a push layer; (ii) drug layer comprising a metformin-transport moiety complex; (iii) a semipermeable wall provided around the push layer and the drug layer; and (iv) an exit.

11. The dosage form of claim 9, comprised of (i) a semipermeable wall provided around an osmotic formulation comprising a metformin-transport moiety complex, an osmagent, and an osmopolymer; and (ii) an exit.

12. The dosage form of claim 9, wherein the dosage form provides a total daily dose of between 500-2550 mg.

13. An improvement in a dosage form comprising metformin or a salt of metformin, the improvement comprising a dosage form comprised of a complex of metformin and a transport moiety.

14. The improved dosage form of claim 13, wherein said transport moiety, prior to complex formation, is a fatty acid of the form CH₃(CₙH₂ₙ)COOH, where n is from 4-16.

15. The improved dosage form of claim 14, wherein said fatty acid is capric acid or lauric acid.

16. A method for treating hyperglycemia in a subject, comprising:
administering the composition of claim 4.

17. The method of claim 16, wherein said administering is via oral administration.

18. A method of preparing a metformin-transport moiety complex, comprising
providing metformin base;
providing a transport moiety;
combining the metformin base and the transport moiety in the presence of a solvent having a dielectric constant less than that of water;
whereby said combining forms a complex comprised of the metformin base and the transport moiety.

19. The method of claim 18, wherein said combining comprises contacting in a solvent having a dielectric constant at least two fold lower than the dielectric constant of water.

20. The method of claim 19, wherein said solvent is selected from the group consisting of methanol, ethanol, acetone, benzene, methylene chloride, and carbon tetrachloride.

21. A method of improving G.I. absorption of metformin, comprising
providing a complex comprised of metformin and a transport moiety, said complex characterized by a tight-ion pair bond; and
administering the complex to a patient.

22. The method of claim 21, wherein the improved absorption comprises improved lower gastrointestinal absorption.

23. The method of claim 21, wherein the improved absorption comprises improved absorption in the upper gastrointestinal tract.

24. A method of treating a subject having Type II diabetes, comprising
administering a complex comprised of metformin and a transport moiety;
administering a second therapeutic agent.

25. The method of claim 24, wherein said administering a second therapeutic agent comprises administering a second therapeutic agent that is an anti-diabetic agent.

26. The method of claim 25, wherein said administering a second therapeutic agent comprises administering a dipeptidyl peptidase IV inhibitor.

27. The method of claim 24, wherein said administering includes administering a complex of metformin and a fatty acid transport moiety, said fatty acid prior to complex formation having the form CH₃(CₙH₂ₙ)COOH, where n is from 4-16.

28. The method of claim 27, wherein said fatty acid is capric acid or lauric acid.

29. The method of claim 24, wherein said administering of the complex includes orally administering the complex.

30. The method of claim 27, wherein said oral administration is achieved by orally administering the complex in an osmotic dosage form.

31. The method of claim 30, comprised of (i) a push layer; (ii) drug layer comprising a metformin-fatty acid complex; (iii) a semipermeable wall provided around the push layer and the drug layer; and (iv) an exit.

32. The method of claim 30, comprised of (i) a semipermeable wall provided around an osmotic formulation comprising a metformin-fatty acid complex, an osmagent, and an osmopolymer; and (ii) an exit.

33. The method of claim 29, wherein the dosage form provides a total daily dose of between 500-2550 mg.

34. The method of claim 24, wherein said administering of the DPP IV inhibitor is via oral administration.

35. A compound comprising metformin and a transport moiety, said compound prepared by a process of (i) providing metformin base; (ii) providing a transport moiety; (iii) combining the metformin base and the transport moiety in the presence of a solvent having a dielectric constant less than that of water, where said combining forms a complex between the metformin base and the transport moiety associated by a tight-ion pair bond.

36. The compound according to claim 35, wherein said transport moiety is a fatty acid of the form CH₃(CₙH₂ₙ)COOH, where n is from 4-16.

37. The compound of claim 36, wherein said fatty acid is capric acid or lauric acid.
